(19) United States
(12) Patent Application Publication
(10) Pub. No.: US 2017/0062015 A1
(43) Pub. Date: Mar. 2, 2017

(54) CORRELATION OF MEDIA WITH BIOMETRIC SENSOR INFORMATION

(71) Applicant: Whole Body IQ, Inc., Monte Sereno, CA (US)

(72) Inventors: Paul J. Zuhorski, Monte Sereno, CA (US); Marianne C. Zuhorski, Monte Sereno, CA (US); Gregory L. Wade, San Jose, CA (US)

(21) Appl. No.: 15/254,893

(22) Filed: Sep. 1, 2016

Related U.S. Application Data
(60) Provisional application No. 62/212,659, filed on Sep. 1, 2015.
(60) Provisional application No. 62/215,851, filed on Sep. 9, 2015.

(51) Int. Cl. G11B 27/34; G11B 27/36; G11B 27/11; A61B 5/00; A61B 5/024

(52) U.S. Cl. CPC G11B 27/34; A61B 5/7246; A61B 5/743; A61B 5/0022; A61B 5/02438; A61B 5/486; G11B 27/11; G11B 27/36

(57) ABSTRACT

Embodiments disclosed herein provide systems, methods, and computer readable media for correlating media with biometric sensor information. In a particular embodiment, a method provides determining that media is being presented to a user. The method also provides receiving biometric sensor information regarding the user captured by at least one biometric sensor during presentation of the media. The method further provides associating portions of the biometric sensor information with contemporaneous portions of the media

### RELATED APPLICATIONS

This application is related to and claims priority to U.S. Provisional Patent Application 62/212,659, titled “CORRELATION OF MEDIA WITH BIOMETRIC SENSOR INFORMATION,” filed Sep. 1, 2015, U.S. Provisional Patent Application 62/215,851, titled “BID BASED SELECTION OF CLOUD COMPUTING PROVIDERS,” filed Sep. 9, 2015, and which are hereby incorporated by reference in their entirety.

### TECHNICAL BACKGROUND

Many products now exist that can track the activity of a user. For example, an activity tracker wristband may be worn by a user to track the user's steps throughout the day. Other devices may include sensors to track the user's heart rate, body temperature, or any other type of biometric information that may be relevant to a user. If a user participates in a workout, the user may use the sensor information to provide details about their performance during the workout, such as whether their heart rate reached a desired level. While this sort of reference information alone may be beneficial to the user, the user may benefit from more specific information, such as what specific activity was being performed during when their heart rate was at any given level.

### OVERVIEW

Embodiments disclosed herein provide systems, methods, and computer readable media for correlating media with biometric sensor information. In a particular embodiment, a method provides determining that media is being presented to a user. The method also provides receiving biometric sensor information regarding the user captured by at least one biometric sensor during presentation of the media. The method further provides associating portions of the biometric sensor information with contemporaneous portions of the media.

In some embodiments, determining that the media is being presented to the user comprises optically capturing a visual indicator presented in the media and processing the visual indicator to identify the media from a plurality of media selections available for correlation.

In some embodiments, processing the visual indicator to identify the media identifies the particular instance of the media being presented to the user.

In some embodiments, the visual indicator comprises a Quick Response (QR) code.

In some embodiments, determining that the media is being presented to the user comprises capturing audio of the presented media and processing the audio to identify the media from a plurality of media selections available for correlation.

In some embodiments, associating the portions of the biometric sensor information with the contemporaneous portions of the media comprises mapping runtime of the media to time of day and mapping capture times of the portions of biometric sensor information to time of day.

In some embodiments, associating the portions of the biometric sensor information with the contemporaneous portions of the media comprises, upon receiving each of the portions of the biometric sensor information, identifying where in runtime of presentation of the media substantially corresponds to a time when the portion of biometric sensor information was captured.

In some embodiments, the time when the portion of biometric sensor information was captured equates to the time when the portion of biometric sensor information was received.

In some embodiments, presenting a graphical representation of portions of the biometric sensor information with contemporaneous portions of the media.

In some embodiments, the biometric sensor information includes a heartrate of the user.

In another embodiment, a system is provided having one or more computer readable storage media and a processing system operatively coupled with the one or more computer readable storage media. Program instructions stored on the one or more computer readable storage media, when read and executed by the processing system, direct the processing system to determine that media is being presented to a user. The program instructions further direct the processing system to receive biometric sensor information regarding the user captured by at least one biometric sensor during presentation of the media and associate portions of the biometric sensor information with contemporaneous portions of the media.

### BRIEF DESCRIPTION OF THE DRAWINGS

Many aspects of the disclosure can be better understood with reference to the following drawings. While several implementations are described in connection with these drawings, the disclosure is not limited to the implementations disclosed herein. On the contrary, the intent is to cover all alternatives, modifications, and equivalents.

FIG. 1 illustrates a media presentation environment for correlating media with biometric sensor information.

FIG. 2 illustrates an operation of the media presentation environment to correlate media with biometric sensor information.

FIG. 3 illustrates an example association of biometric sensor information generated during the operation of the media presentation environment to correlate media with biometric sensor information.

FIG. 4 illustrates another media presentation environment for correlating media with biometric sensor information.

FIG. 5 illustrates an operation of the other media presentation environment to correlate media with biometric sensor information

FIG. 6 illustrates a biometric correlation system for correlating media with biometric sensor information.

FIG. 7 illustrates a cloud computing environment in an example scenario for selecting cloud computing providers.

FIG. 8 illustrates an operation of the cloud computing environment in an example scenario for selecting cloud computing providers.

FIG. 9 illustrates an example visualization of an operational scenario for selecting cloud computing providers.

FIG. 10 illustrates a cloud provider selection system in an example scenario for selecting cloud computing providers

### DETAILED DESCRIPTION

Many types of sensors may be used to obtain biometric information about a user. In fact, sensors that used to be bulky, such as heart rate monitors, are now available to in form factors, such as bracelets, that allow a user to wear those sensors more frequently. Therefore, when a user is working out or even simply going through their daily activities, biometric information about the user may be easily obtained. For example, various biometric sensors, such as accelerometers/pedometers, heart rate monitors, Global Positioning System (GPS) receivers, and the like, can track a user's steps, heart rate, body temperature, motion, etc. More advanced sensors may even be able to track information such as blood glucose levels, blood oxygen levels, or any other type of biometric information.

In many cases, like when a user is working out, media that is being presented to a user may impact the user's biometric information. For example, a workout instructional video providing the user with an exercise routine will likely effect the user's biometric information as the user progresses through the routine. Alternatively, the media may less overtly effect the user's biometric information, as may be the case when the beat of a song increases or decreases. Regardless of the media type or the activity performed by a user when presented with that media, the embodiments described below correlate the biometric information received during media presentation to points (or time periods) in that media presentation. A user, or anyone else having access to the correlation information, can then determine what portions of biometric information correspond to what portions of the media.

FIG. 1 illustrates media presentation environment 100 in an example scenario for correlating media with biometric sensor information. Media presentation environment 100 includes biometric correlation system 101, media presentation system 102, and biometric sensors 103. Biometric correlation system 101 and media presentation system 102 communicate over communication link 111. Biometric correlation system 101 and biometric sensors 103 communicate over communication link 112.

While shown separately, media presentation system 102 and biometric correlation system 101 may be incorporated into the same device, such as a smartphone, tablet, or personal computer. Biometric sensors 103 may be worn by user 121, implanted into user 121, or otherwise positioned in a manner suitable to measure biometric information of user 121. Biometric sensors 103 may be included in a single device or distributed across multiple devices, such as smartphones, smart watches, fitness trackers, dedicated sensor devices, or some other type of device. Likewise, one or more of biometric sensors 103 may be incorporated into one or more of media presentation system 102 and biometric correlations system 101. In some examples, such as a smart watch or smartphone, at least some of the functionality of all three elements may be incorporated into a single device.

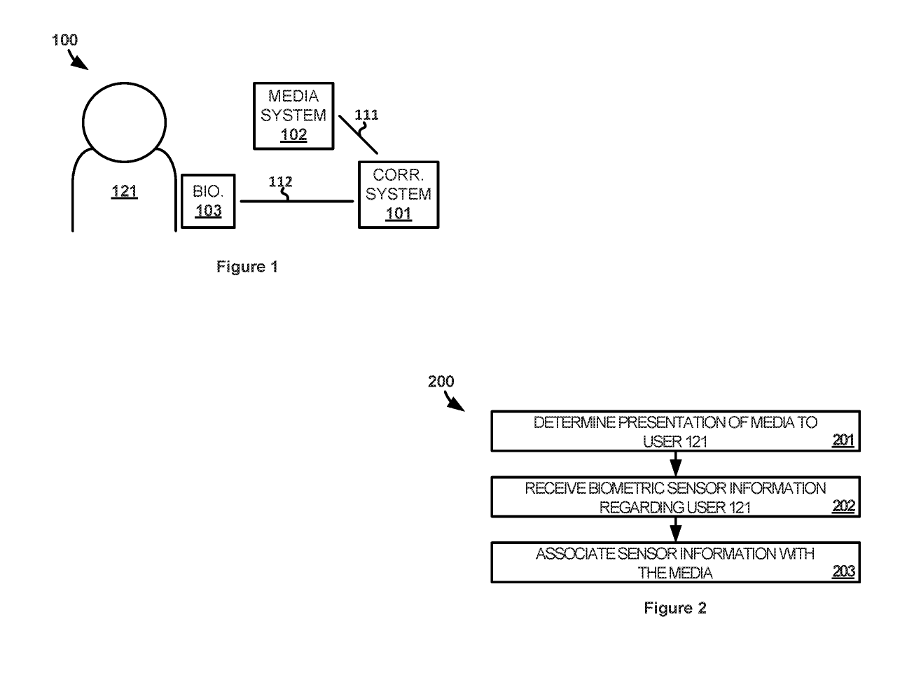

FIG. 2 illustrates operation 200 of media presentation environment 100 to correlate media with biometric sensor information. Operation 200 provides biometric correlation system 101 determining that media is being presented to user 121 (step 201). In this example, the media is presented to user 121 by media presentation system 102, which may be a television monitor, computer, tablet, smartphone, personal media player, or some other type of device capable of presenting audible and/or visual media to a user. The media may be presented through a website displayed by a web browser running on system 102, by a media player application running on system 102, or by some other means.

Biometric correlation system 101 may determine that the media is being presented to user 121 by receiving information identifying the media (e.g. a particular video provided by a website) and receiving an indication when the media begins playing indicated such. Subsequent indications may be received by biometric correlation system 101 to ensure biometric correlation system 101 is informed of the current position in the presentation of the media (e.g. when the media is paused, fast forwarded, rewound, etc.). This information may be obtained by biometric correlation system 101 using application programming interfaces (APIs) in an interface (e.g. application, website, etc.) for presenting the media on media presentation system 102, APIs for biometric correlation system 101, or some other means for biometric correlation system 101 to ascertain media presentation information—including combinations thereof. In a particular example, a Quick Response (QR) code or the like may be displayed by the media. That code may be optically captured by a device (e.g. biometric correlation system 101 or some other device in communication with system 101). The information in that code may then be used by biometric correlation system 101 to identify the media (along with any timing, activity, etc. information that may be relevant to operation 200) and associate the media with biometric sensor information in the steps below. In some examples, such as media presented via a website, the presentation information may be obtained from a server (e.g. web server for the website) rather than from media presentation system 102 directly.

Operation 200 further provides that biometric correlation system 101 receives biometric sensor information regarding user 121 captured by at least one of biometric sensors 103 during presentation of the media (step 202). In some examples, biometric sensors 103 may transfer captured biometric information for receipt by biometric correlation system 101 in real time or may transfer the biometric information at some later time, either at the instruction of user 121 (e.g. when biometric sensors 103 are synchronized with biometric correlation system 101), automatically, or otherwise. The biometric information may include time stamps to indicate when portions of the biometric information was captured. The biometric sensor information may include heart rate, location information (e.g. GPS information), speed information (e.g. derived from the GPS or provided by exercise equipment), elevation information, pedometer information, blood glucose levels, or any other type of information related to user 121's activity. Biometric correlation system 101 may only receive the biometric sensor information collected while the media is being presented (e.g. begins collecting the sensor information when the media begins playing) or may also receive the sensor information at other times (e.g. for purposes other than correlation with the media). Likewise, sensor information may be collected periodically, continually, or upon some trigger, which may depend on how many data points biometric correlation system 101 requires to perform step 203, as disclosed below.

Operation 200 also provides biometric correlation system 101 associating portions of the biometric sensor information with contemporaneous portions of the media (step 203). The association may be performed in real-time during presentation of the media or may be performed at a later time. In some examples, biometric correlation system 101 associates specific points in the media with the biometric sensor information that was captured at that time. For instance, a video being played may reach the 2 minute and 35 second mark at 9:38:23 AM. The associated portion of the biometric sensor information to that point in the video would therefore comprise the sensor information received at or substantially near (e.g. the first sensor reading occurring after) 9:38:23 AM. Biometric correlation system 101 may perform the association using time stamps in the media and/or the sensor information or may rely on when the sensor information is received following a certain point in the media. In alternative examples, the sensor information may be provided for a span of time during the media presentation. For instance, the sensor information may be associated with one minute portions of a video and, therefore, the portion of the sensor information may be an average of sensor information received over that one-minute period. In yet more examples, the portions of sensor information may correspond to a certain activity in the media, such as running, walking, pushups, sit-ups, jumping jacks, weight lifting, or any other type of activity. The portions of the media corresponding to each activity may be provided by the media supplier (e.g. using metadata in the media or otherwise), by description entered by user 121, or from some other source. A portion of the sensor information corresponding to each activity will therefore be associated with the activity's portion of the media. Other variations on associating portions of sensor information with portions of the media may also be used.

Once associated, the associations may be stored for later access (e.g. in a separate file, within metadata of the media, as part of an application, or otherwise). User 121, another user, a health provider, or otherwise, may then use the associations for various purposes, such as simply viewing the information through a graphical interface or for analysis either on their own or along with other sets of associations (e.g. to generate averages, medians, etc.). For example, the associations may be used to derive a fitness level for user 121 from the biometric sensor information associated with various activities provided for in the media. Moreover, previous associations from operation 200 may be included in the fitness analysis to track the fitness progress of user 121 over time. Likewise, associations of biometric sensor information from other users with the same media may be used to determine a fitness level of user 121 relative to other users with respect to the same media. In further examples, QR codes may be used to categorize workouts that are presented in various media items so that comparisons can easily be made with relevant historical associations generated using operation 200.

While the example above collects and associates biometric information of a single user with the media, it should be understood that in some examples multiple users may be viewing the same media presentation from system 102 and biometric information may be associated with the media for each of those users as well.

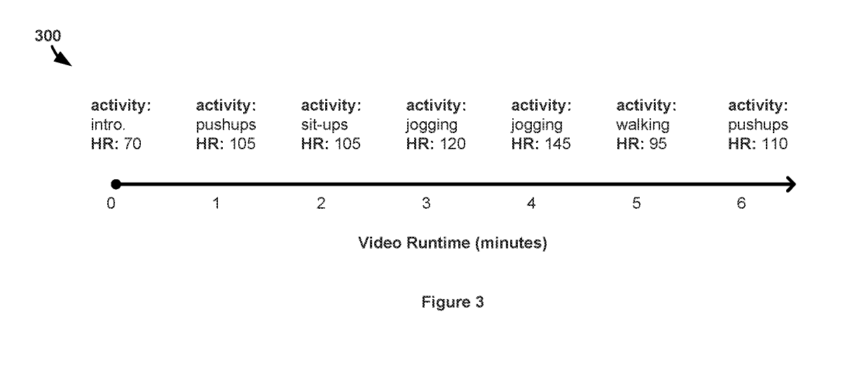

FIG. 3 illustrates an example visualization 300 of an association of biometric sensor information generated during the operation of media presentation environment 100 to correlate media with biometric sensor information. In this example, the media is an exercise video that instructs a user through a workout. Biometric information is associated with the video at every minute during the playback of the video beginning with when the video playback is started. For simplicity, the only biometric information collected for user 121 in this example is their heart rate at each point in the video runtime. Additionally, while visualization 300 shows the activity being performed at each minute interval, the activity may have to be gleaned from watching the video rather than being included in the associations.

In some examples, sensor information begins being collected by biometric correlation system 101 when the user instructs media system 102 to start playing the video. In more specific examples, a separate instruction (e.g. a separate on screen play button) may be used to indicate that the user wishes to have biometric information captured during the video, as opposed to playing the video without collecting information.

Once playback starts, biometric correlation system 101 starts receiving heart rate information from a heart rate sensor of sensors 103. As can be seen in visualization 300, user 121's heart rate is at 70 beats per minute (BPM) when the video starts and an introduction is being played. At minute 1, user 121 is doing pushups as instructed by the video and their heart rate has increased to 105 BPM, which is the same heart rate as when user 121 is doing sit-ups at minute 2. The heart rate goes up to 120 when user 121 begins jogging at minute 3 and increases to 145 as jogging progresses to minute 4. The heart rate goes back down to 95 when walking at minute 5 and then back up to 110 when user 121 does more pushups. While visualization 300 stops at minute 6, the video may continue beyond the 6 minutes of this example.

Based on the associations shown in visualization 300, it can easily be determined what user 121's heart rate was during any given activity. In more complex examples, the biometric sensor information may include counts as to how many pushups or sit-ups user 121 performed during the video, how far user 121 ran when jogging (e.g. received from a treadmill user 121 ran on), how many steps user 121 took, or any other possible type of biometric information that could be received by biometric correlation system 101 from sensors 103. Likewise, user 121 may be able to augment the biometric sensor information with their own input, such as a description of how user 121 felt during each activity.

Referring back to FIG. 1, biometric correlation system 101 comprises a computer system and communication interface. Biometric correlation system 101 may also include other components such as a router, server, data storage system, and power supply. Biometric correlation system 101 may reside in a single device or may be distributed across multiple devices. Biometric correlation system 101 could be an application server(s), a personal computer, tablet, smartphone, watch, media player, television, or some other computing system—including combinations thereof.

Media presentation system 102 comprises a computer system and communication interface. Media presentation system 102 may also include other components such as a router, server, data storage system, and power supply. Media presentation system 102 may reside in a single device or may be distributed across multiple devices. Media presentation system 102 could be an application server(s), a personal computer, tablet, smartphone, watch, media player, television, or some other computing system—including combinations thereof.

Communication links 111 and 112 could be internal system busses or use various communication protocols, such as Time Division Multiplex (TDM), Internet Protocol (IP), Ethernet, communication signaling, Code Division Multiple Access (CDMA), Evolution Data Only (EVDO), Worldwide Interoperability for Microwave Access (WIMAX), Global System for Mobile Communication (GSM), Long Term Evolution (LTE), Wireless Fidelity (WIFI), Bluetooth, High Speed Packet Access (HSPA), or some other communication format—including combinations thereof. Communication links 111 and 112 could be direct links or may include intermediate networks, systems, or devices.

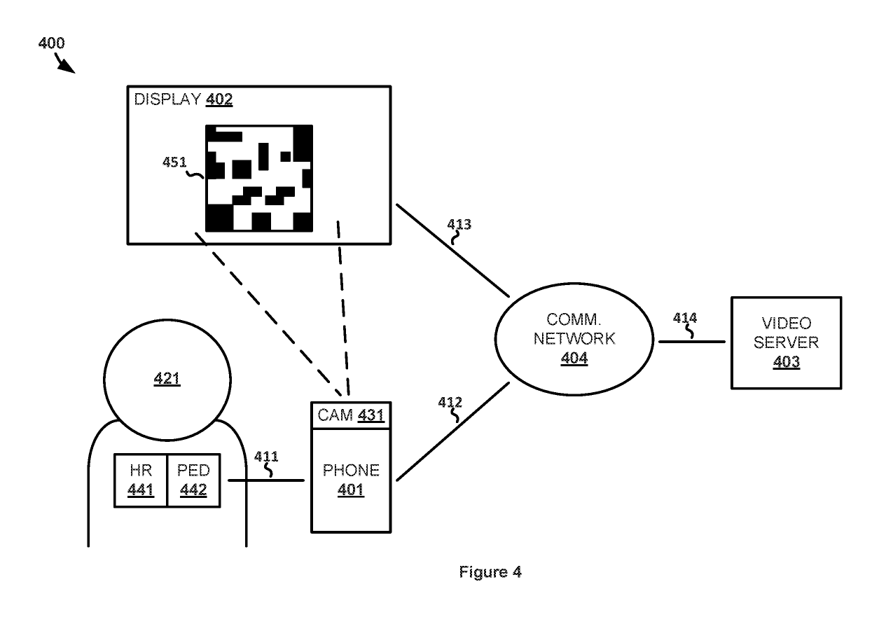

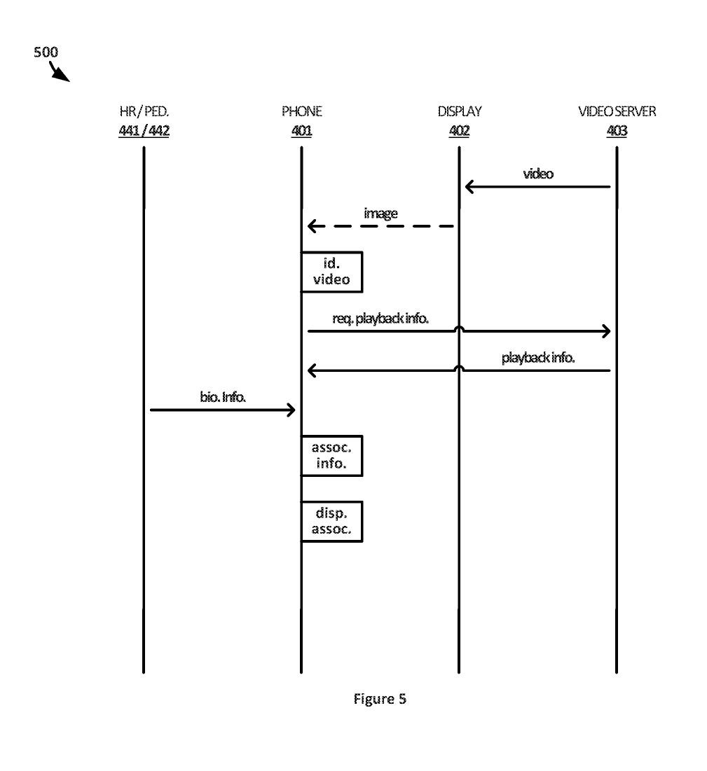

FIG. 4 illustrates media presentation environment 400 in an example scenario for correlating media with biometric sensor information. Media presentation environment 400 includes smartphone 401, display 402, video server system 403, communication network 404, heartrate monitor 441, and pedometer 442. Smartphone 401 and communication network 404 communicate over communication link 412. Display 402 and communication network 404 communicate over communication link 413. Video server system 403 and communication network 404 communicate over communication link 414. Smartphone 401 and heartrate monitor 441/pedometer 442 communicate over communication link 411.

In operation, the functionality of biometric correlation system 101 is built into smartphone 401. Heartrate monitor 441 and pedometer 442 are in communication with smartphone 401 to provide heartrate and step information to smartphone 401 about user 421. In one example, heartrate monitor 441 and pedometer 442 may be included in an accessory like a smartwatch, although, heartrate monitor 441 and pedometer 442 may take other form factors and may exist in separate devices. In those examples, communication link 411 may comprise a Bluetooth link or may communicate over communication network 404 instead of directly with smartphone 401.

Display 402, as shown, may comprise a video panel having the processing and communication circuitry required to retrieve and playback video media supplied by video server system 403. In alternative examples, display 402 may be coupled with a media player system that retrieves video media from video server system 403 and provides a video signal (and likely an accompanying audio signal) for display 402 to present.

FIG. 5 illustrates operation 500 of media presentation environment 400 to correlate media with biometric sensor information. In operation 500, a video is received from video server system 403 and displayed by video server system 403. The video may be selected by user 421 via display 402, via smartphone 401 in communication with display 402 or video server system 403, or in some other manner in which a video can be selected. The video may be selected from a catalog of videos that are compatible for biometric correlation. Videos in the catalog may include descriptions of the video at given points in the runtime (e.g. a workout video may indicate what activities are being performed at various points during the video) to provide more context when the video runtime is correlated with biometric information. The video may be streamed to display 402 or delivered in its entirety at some point before the playback of the video.

At the beginning of the video presentation, a Quick Response (QR) code 451 is presented as part of the video. In other examples, a different type of visual indicator may be included in the video or the video itself may be used as a visual indicator without any special purpose indicator like QR code 451. User 421 operates smartphone 401 to capture an image of QR code 451 using camera 431 of smartphone 401. An application may execute on smartphone 401 to capture QR code 451 and correlate biometric information. QR code 451 encodes information identifying the video to smartphone 401. In this case, QR code 451 identifies the specific instance of the video being delivered to display 402 so that smartphone 401 can communicate with video server system 403 to retrieve playback information about the video being presented by display 402 (rather than some other instance of the same video). In some cases, user 421 may need to pause playback of the video in order to keep QR code 451 on the screen long enough to capture. In other examples, audio indicators may be used instead of visual indicators to identify the video. In those examples, a microphone of smartphone 401 may be used to capture audio presented by display 402 as part of the video presentation and that audio is processed to identify the video. Of course, other ways of identifying the video to smartphone 401 may also be used.

Once the video is identified, smartphone 401 requests playback information from video server system 403. Video server system 403 then provides playback information to smartphone 401. For instance, video server system 403 may provide indications of when the video is playing, when it is paused, when it is rewound or fast forwarded, or any other information that can be used by smartphone 401 to determine when the video playback was at a given point in runtime. In one example, smartphone 401 maps runtime of the video to time of day so that when biometric information is captured, that can also be mapped to time of day for easier association. Thus, when smartphone 401 begins receiving heartrate and pedometer information from heartrate monitor 441 and pedometer 442, smartphone 401 can associate instances when such information was captured with points in the runtime of the video. The association may be performed as the information is captured and the video plays or may be associated at a later point, such as when the video is finished playing.

The associations can then be displayed by smartphone 401 so that user 421 can visualize their biometrics as they were captured during the playback of the video. The associations may be displayed in a timeline similar to visualization 300, although, different manners of displaying the associations may also be used. In some cases, additional information may be displayed as well, such as screen shots of the video at given points, descriptive information about what is occurring in the video at given points, or any other type of information that might be helpful for user 421 to analyze their biometric information. Advantageously, through operation 500, user 421 is able to track biometric information of a workout or other type of activity in conjunction with a video played during that activity.

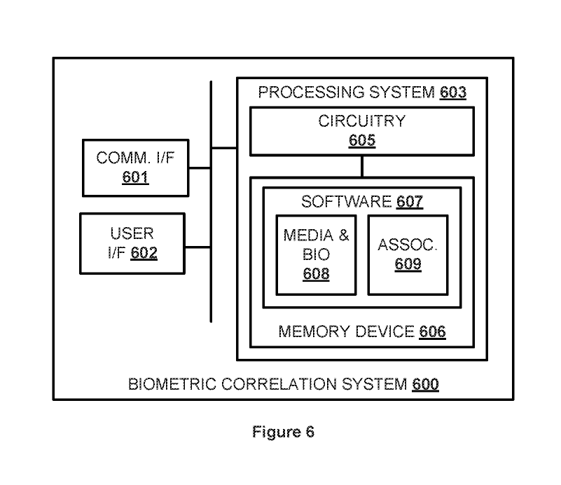

FIG. 6 illustrates biometric correlation system 600. Biometric correlation system 600 is an example of biometric correlation system 101, although system 101 may use alternative configurations. Biometric correlation system 600 comprises communication interface 601, user interface 602, and processing system 603. Processing system 603 is linked to communication interface 601 and user interface 602. Processing system 603 includes processing circuitry 605 and memory device 606 that stores operating software 607.

Communication interface 601 comprises components that communicate over communication links, such as network cards, ports, RF transceivers, processing circuitry and software, or some other communication devices. Communication interface 601 may be configured to communicate over metallic, wireless, or optical links Communication interface 601 may be configured to use TDM, IP, Ethernet, optical networking, wireless protocols, communication signaling, or some other communication format—including combinations thereof.

User interface 602 comprises components that interact with a user. User interface 602 may include a keyboard, display screen, mouse, touch pad, or some other user input/output apparatus. User interface 602 may be omitted in some examples.

Processing circuitry 605 comprises microprocessor and other circuitry that retrieves and executes operating software 607 from memory device 606. Memory device 606 comprises a non-transitory storage medium, such as a disk drive, flash drive, data storage circuitry, or some other memory apparatus. Operating software 607 comprises computer programs, firmware, or some other form of machine-readable processing instructions. Operating software 607 includes media presentation and biometric information module 608 and biometric information association module 609. Operating software 607 may further include an operating system, utilities, drivers, network interfaces, applications, or some other type of software. When executed by circuitry 605, operating software 607 directs processing system 603 to operate Biometric correlation system 600 as described herein.

In particular, media presentation and biometric information module 608 directs processing system 603 to determine that media is being presented to a user and, during presentation of the media, to receive biometric sensor information regarding the user from at least one biometric sensor. Biometric information association module 609 directs processing system 603 to associate portions of the biometric sensor information with contemporaneous portions of the media.

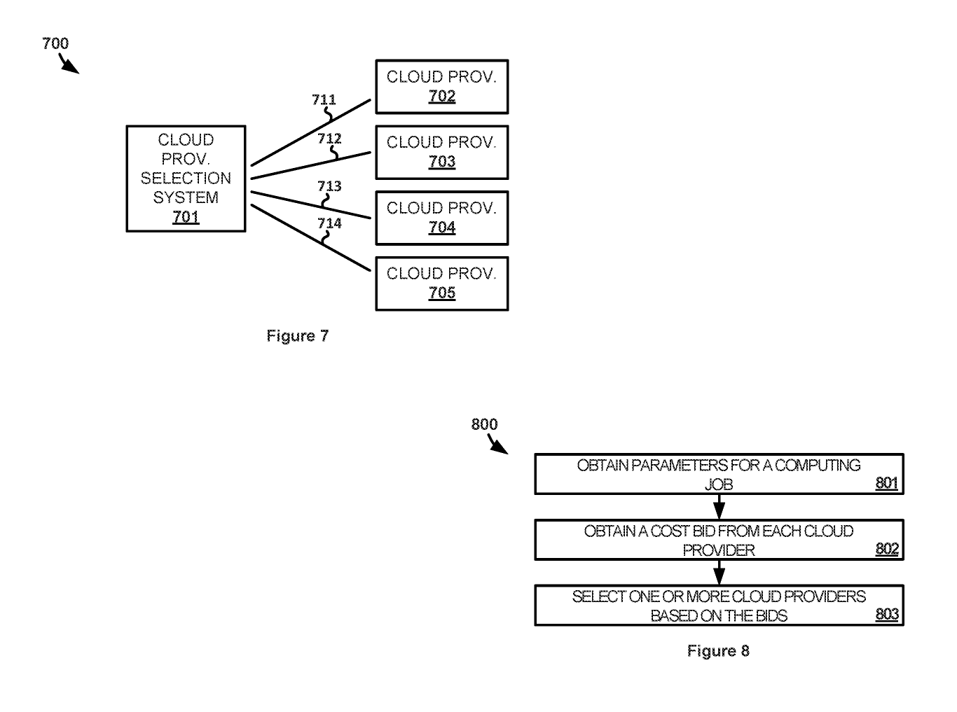

FIG. 7 illustrates cloud computing environment 700 in an example scenario for selecting cloud computing providers. Cloud computing environment 700 includes cloud provider selection system 701 and cloud computing providers 702-705. Cloud computing environment 700 and cloud computing providers 702-705 communicate over communication links 711-714, respectively.

Cloud computing providers sell computing resources, such a processor time, memory, storage, and bandwidth, among other computing resources, to others that do not have or are otherwise unable to perform data tasks (e.g. data processing jobs, data storage, etc.) using their own resources. These other entities (e.g. individuals, companies, universities, etc.) are therefore able to purchase computing resources from the cloud service providers to perform the desired data task. As these cloud computing resources become more of a commodity sold by the cloud computing providers, it is more likely that the cost (e.g. time cost, monetary cost, etc.) of the cloud computing resources will matter more to an entity than the particular provider that is selling the resources.

Cloud computing providers 702-705 provide computing resources to any entity that is able to pay for those resources. While four cloud computing providers are shown in environment 700, cloud provider selection system 701 may have any number of cloud computing providers to select from.

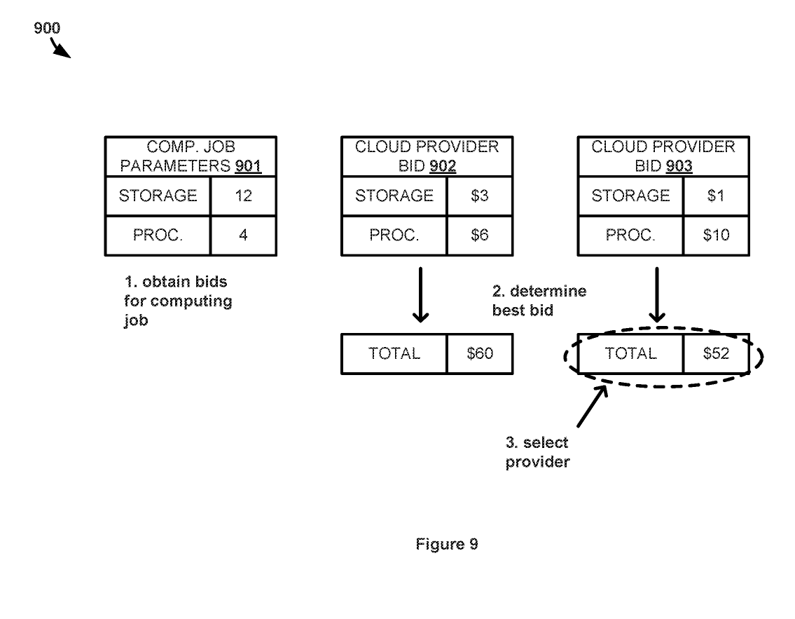

FIG. 8 illustrates operation 800 of cloud computing environment 700 to select cloud computing providers. Operation 800 provides that cloud provider selection system 701 obtains one or more parameters for a computing job (step 801). The parameters may be received via user input into cloud provider selection system 701, received via user input into another system before being transferred to cloud provider selection system 701 (e.g. via a network), generated by cloud provider selection system 701 after analyzing the computing job, generated by another system before being transferred to cloud provider selection system 701, or any other way by which a system may receive such information.

The parameters may include an amount of bandwidth to transfer data (e.g. to or from the cloud provider), amount of primary and/or secondary data storage, amount random access memory (RAM), a number of processor units, an amount of processor time, an amount of wall time, an amount of Input/Output Operations Per Second (IOPS), or any other type of parameter that a computing job may have—including combinations thereof. Some computing jobs may have many parameters while other computing jobs may have only one, which may simply be a parameter indicating that the job needs to be done. The parameters may include one or more cost thresholds, such as monetary cost or time cost, or may define cost in more broad manner, such as indicating the lowest possible cost. Likewise, the parameters may include priorities for other parameters. For example, if the computing job needs to be completed quickly, the wall time required to complete the computing job may be prioritized over the monetary cost of completion (possibly only to an extent).

In further examples, the parameter for the job include both mandatory and flexible parameters. For instance, the amount of storage needed may be mandatory while the processing time may be flexible. While a cloud computing provider that meets all parameters would be ideal, the flexible parameters allow for variation when providing bids. In some cases, the flexible parameters may be prioritized over one another. Thus, bids can then be evaluated by cloud provider selection system 701 based on how close the corresponding cloud computing provider can come to higher priority flexible parameters over lower priority flexible parameters. In some cases, the amount of variation from the desired flexible parameter may depend on the cost of that variation. That is, even if a certain bid is relatively far outside of the desired flexible parameter, that bid may be low enough in cost to account for that variation.

Cloud provider selection system 701 further obtains a cost bid based on the one or more parameters from each of cloud computing providers 702-705 (step 802). The cost bids may be requested from each of cloud computing providers 702-705 by cloud provider selection system 701, may be transferred by computing providers 702-705 to cloud provider selection system 701 periodically or upon a cost changing, or by some other means. In some examples, cloud computing providers 702-705 may provide application programming interfaces (APIs) that cloud provider selection system 701 uses to obtain the costs. The cost bids from each of cloud computing providers 702-705 may indicate an overall cost to satisfy the parameters for the computing job or may indicate the cost of each parameter individually. In some examples, the cost bids may merely indicate a current cost of each resource provided by the particular cloud computing provider, which would allow cloud computing providers 702-705 to calculate the costs for the parameters of the computing job. The bids from certain cloud computing providers in some cases may include incentives for loyalty, which the cloud computing providers may use to differentiate themselves from others.

Once the bids are obtained, cloud provider selection system 701 selects one or more of cloud computing providers 702-705 based on the cost bids (step 803). A single cloud computing provider may be selected if that one cloud computing provider is able to satisfy all the parameters or at least satisfy the parameters to a greater extent than other cloud computing providers. Alternatively, if it is possible for the cloud computing job to be separated among multiple cloud computing providers, as may be indicated in the parameters, multiple cloud computing providers may be selected to best satisfy the parameters. For example, for a data processing job to be performed within an amount of wall time defined by the parameters, portions of that data processing job, if possible, may be divided among different cloud computing providers. In an alternative example, maybe one cloud computing provider is selected to process data while another is selected to store the resulting data.

As noted above, each of the parameters for a computing job may be prioritized differently. Therefore, when selecting the cloud computing providers 702-705, cloud provider selection system 701 will select a cloud computing provider that satisfies higher prioritized parameters even if lower priority parameters are not satisfied. For example, if a wall time for completing a data process is the highest priority parameter and only one of cloud computing providers 702-705 satisfy that parameter, then cloud provider selection system 701 will select that one cloud computing provider even if it does not satisfy a monetary cost parameter. Conversely, if the monetary cost parameter is higher priority, then a cloud computing provider that best satisfies the monetary cost parameter (or gets the closest to satisfying that parameter) may be selected over the cloud computing provider that satisfies the wall time parameter.

In some examples, the parameters and bids may be used to determine what type of computing job should be used in a particular situation. For example, data may be processed in a first computing job that produces resulting data. It may be known or anticipated that the resulting data will need to be used again in the future, therefore a parameter may indicate a time period during which the resulting data needs to be stored in the cloud computing platform. However, the bids may indicate to cloud provider selection system 701 that a lower monetary cost would be incurred if the data is retransferred (i.e. bandwidth cost) and reprocessed (i.e. processor cost) by a cloud computing platform to regenerate the resulting data than would be incurred if the resulting data is stored for the required time period (i.e. storage cost).

Once cloud provider selection system 701 has selected the cloud computing providers, cloud provider selection system 701 may transfer the computing job to the selected cloud computing providers, instruct another system to transfer the computing job to the selected cloud computing provider, inform a user or system of the selection, or perform some other action relevant to a selected cloud computing provider.

In some cases, cloud provider selection system 701 will repeat operation 800 to ensure the best cloud service provider is being used for the job, especially in cases when the job in ongoing or is recurs frequently. Likewise, cloud provider selection system 701 may continue to solicit bids based on changes to the job to determine whether the changes will result in more cost effective usage of a cloud computing provider.

FIG. 9 illustrates an example visualization of an operational scenario 900 for selecting cloud computing providers. Computing job parameters 901 show cloud computing resources that a computing job will need to run in a cloud computing provider. Specifically, job parameters 901 indicate that the job will require 12 storage units and 4 processing units. The storage units may comprise any unit of storage resources and may include a unit of time parameter. For example, a storage unit may be defined as one terabyte stored for one month. Likewise, processing units may comprise any unit of processing resources and may also include a unit of time. For example, a processing unit may be defined as one processor element (e.g. core) for one hour. Thus, using that definition, the 4 processing units of computing job parameters 901 may be satisfied by one processor element running for 4 hours, two processor elements running for 2 hours each, four processor elements running for one hour each, or some other possible combination that would satisfy the 4 processing unit parameter. Of course, those combinations may be limited based on whether the computing job associated with computing job parameters 901 can be parallelized in that manner

At step 1, cloud provider selection system 701 obtains bids from cloud computing providers to select a cloud computing provider that can satisfy computing job parameters 901. In this example, only two bids are obtained, cloud provider bid 902 and cloud provider bid 903, which are obtained from cloud computing provider 702 and cloud computing provider 703, respectively. Bids are not obtained from cloud computing provider 704 and cloud computing provider 705 for simplicity of this example. Additionally, the cloud provider bid 902 and cloud provider bid 903 indicate an example cost per unit in dollars. In other examples, cloud provider selection system 701 may provide cloud computing providers 702 and 703 with computing job parameters 901 and receive bids from each that indicate a cost for either or both parameters or a cost for the entire computing job so that cloud provider selection system 701 does not need to calculate total cost itself.

At step 2, cloud provider selection system 701 determines which of the two cloud provider bids 902 and 903 is the best for the purposes of running the computing job. Thus, cloud provider selection system 701 calculates that total cost for the computing job that satisfies computing job parameters 901. Cloud provider selection system 701 multiplies the cost for each storage unit indicated in cloud provider bids 902 and 903 by the 12 storage units specified by computing job parameters 901. Similarly, cloud provider selection system 701 multiplies the cost for each processing unit indicated in cloud provider bids 902 and 903 by the 4 processing units specified by computing job parameters 901. Those two multiplication results are added together to find that cloud provider bid 902 would cost $60 total for the computing job and cloud provider bid 903 would cost $52 total for the computing job.

Accordingly, assuming that both cloud computing providers will provide the same results, cloud provider selection system 701 selects the cloud computing provider with the cheapest bid at step 3, which cloud computing provider 703 having cloud provider bid 903. In another example, assuming that the bandwidth cost to transfer data is negligible, cloud provider selection system 701 may select cloud computing provider 702 for processing units and cloud computing provider 703 for storage units since cloud provider bid 902 has a lower cost for processing units and cloud provider bid 903 has a lower cost for storage units. In yet another example, even though cloud provider bid 902 is more expensive, cloud computing provider 702 may be able to perform the computing job faster. In that example, if a lower wall time is an additional parameter of computing job parameters 901, then cloud provider selection system 701 may select cloud computing provider 702 for the lower wall time.

Referring back to FIG. 7, cloud provider selection system 701 comprises a computer system and communication interface. Cloud provider selection system 701 may also include other components such as a router, server, data storage system, and power supply. Cloud provider selection system 701 may reside in a single device or may be distributed across multiple devices. Cloud provider selection system 701 could be an application server(s), a personal computer, tablet, smartphone, or some other computing system—including combinations thereof.

Cloud computing providers 702-705 each comprise a computer system and communication interface. Cloud computing providers 702-705 may also include other components such as a router, server, data storage system, and power supply. Cloud computing providers 702-705 may reside in a single device or may be distributed across multiple devices. Cloud computing providers 702-705 could include server(s) and data storage systems—including combinations thereof—to provide data processor and storage capacity.

Communication links 711-714 could be internal system busses or use various communication protocols, such as Time Division Multiplex (TDM), Internet Protocol (IP), Ethernet, communication signaling, Code Division Multiple Access (CDMA), Evolution Data Only (EVDO), Worldwide Interoperability for Microwave Access (WIMAX), Global System for Mobile Communication (GSM), Long Term Evolution (LTE), Wireless Fidelity (WIFI), Bluetooth, High Speed Packet Access (HSPA), or some other communication format—including combinations thereof. Communication links 711-714 could be direct links or may include intermediate networks, systems, or devices.

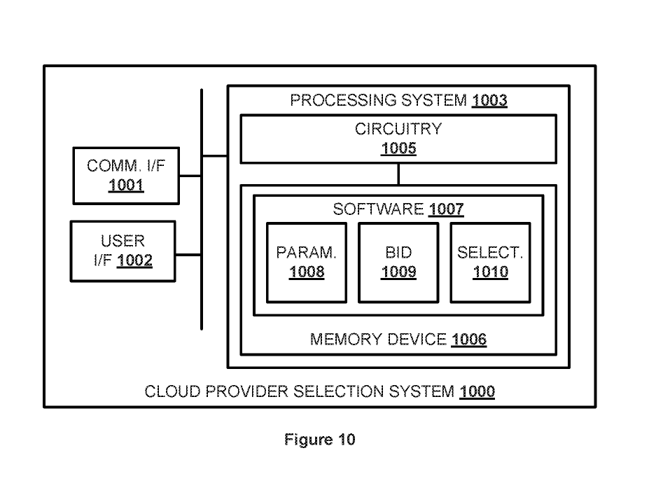

FIG. 10 illustrates cloud provider selection system 1000. Cloud provider selection system 1000 is an example of cloud provider selection system 701, although system 701 may use alternative configurations. Cloud provider selection system 1000 comprises communication interface 1001, user interface 1002, and processing system 1003. Processing system 1003 is linked to communication interface 1001 and user interface 1002. Processing system 1003 includes processing circuitry 1005 and memory device 1006 that stores operating software 1007.

Communication interface 1001 comprises components that communicate over communication links, such as network cards, ports, RF transceivers, processing circuitry and software, or some other communication devices. Communication interface 1001 may be configured to communicate over metallic, wireless, or optical links Communication interface 1001 may be configured to use TDM, IP, Ethernet, optical networking, wireless protocols, communication signaling, or some other communication format—including combinations thereof.

User interface 1002 comprises components that interact with a user. User interface 1002 may include a keyboard, display screen, mouse, touch pad, or some other user input/output apparatus. User interface 1002 may be omitted in some examples.

Processing circuitry 1005 comprises microprocessor and other circuitry that retrieves and executes operating software 1007 from memory device 1006. Memory device 1006 comprises a non-transitory storage medium, such as a disk drive, flash drive, data storage circuitry, or some other memory apparatus. Operating software 1007 comprises computer programs, firmware, or some other form of machine-readable processing instructions. Operating software 1007 includes computing job parameter module 1008, bid module 1009, and provider selection module 1010. Operating software 1007 may further include an operating system, utilities, drivers, network interfaces, applications, or some other type of software. When executed by circuitry 1005, operating software 1007 directs processing system 1003 to operate Cloud provider selection system 1000 as described herein.

In particular, computing job parameter module 1008 directs processing system 1003 to obtain one or more parameters for a computing job. Bid module 1009 directs processing system 1003 to obtain a cost bid based on the one or more parameters from each of the plurality of cloud computing providers. Provider selection module 1010 directs processing system 1003 to select one or more cloud computing providers of the plurality of cloud computing providers based on the cost bids.

The above description and associated figures teach the best mode of the invention. The following claims specify the scope of the invention. Note that some aspects of the best mode may not fall within the scope of the invention as specified by the claims. Those skilled in the art will appreciate that the features described above can be combined in various ways to form multiple variations of the invention. As a result, the invention is not limited to the specific embodiments described above, but only by the following claims and their equivalents.

What is claimed is:

1. A method of correlating media with biometric sensor information, the method comprising:
determining that media is being presented to a user;
receiving biometric sensor information regarding the user captured by at least one biometric sensor during presentation of the media; and
associating portions of the biometric sensor information with contemporaneous portions of the media.

2. The method of claim 1, wherein determining that the media is being presented to the user comprises:
optically capturing a visual indicator presented in the media;
processing the visual indicator to identify the media from a plurality of media selections available for correlation.

3. The method of claim 2, wherein processing the visual indicator to identify the media identifies the particular instance of the media being presented to the user.

4. The method of claim 2, wherein the visual indicator comprises a Quick Response (QR) code.

5. The method of claim 1, wherein determining that the media is being presented to the user comprises:
capturing audio of the presented media;
processing the audio to identify the media from a plurality of media selections available for correlation.

6. The method of claim 1, wherein associating the portions of the biometric sensor information with the contemporaneous portions of the media comprises:
mapping runtime of the media to time of day; and
mapping capture times of the portions of biometric sensor information to time of day.

7. The method of claim 1, wherein associating the portions of the biometric sensor information with the contemporaneous portions of the media comprises:
upon receiving each of the portions of the biometric sensor information, identifying where in runtime of presentation of the media substantially corresponds to a time when the portion of biometric sensor information was captured.

8. The method of claim 7, wherein the time when the portion of biometric sensor information was captured equates to the time when the portion of biometric sensor information was received.

9. The method of claim 1, further comprising:
presenting a graphical representation of portions of the biometric sensor information with contemporaneous portions of the media.

10. The method of claim 1, wherein the biometric sensor information includes a heartrate of the user.

11. A system to correlate media with biometric sensor information, the data management system comprising:
one or more computer readable storage media;
a processing system operatively coupled with the one or more computer readable storage media; and
program instructions stored on the one or more computer readable storage media that, when read and executed by the processing system, direct the processing system to:
determine that media is being presented to a user;
receive biometric sensor information regarding the user captured by at least one biometric sensor during presentation of the media; and
associate portions of the biometric sensor information with contemporaneous portions of the media.

12. The system of claim 11, wherein to determine that the media is being presented to the user, the program instructions direct the processing system to at least:
optically capture a visual indicator presented in the media;
process the visual indicator to identify the media from a plurality of media selections available for correlation.

13. The system of claim 12, wherein the program instructions direct the processing system to process the visual indicator to identify the media identifies the particular instance of the media being presented to the user.

14. The system of claim 12, wherein the visual indicator comprises a Quick Response (QR) code.

15. The system of claim 11, wherein to determine that the media is being presented to the user, the program instructions direct the processing system to at least:
capture audio of the presented media;
process the audio to identify the media from a plurality of media selections available for correlation.

16. The system of claim 11, wherein to associate the portions of the biometric sensor information with the contemporaneous portions of the media, the program instructions direct the processing system to at least:
map runtime of the media to time of day; and
map capture times of the portions of biometric sensor information to time of day.

17. The system of claim 11, wherein to associate the portions of the biometric sensor information with the contemporaneous portions of the media, the program instructions direct the processing system to at least:
upon receiving each of the portions of the biometric sensor information, identify where in runtime of presentation of the media substantially corresponds to a time when the portion of biometric sensor information was captured.

18. The system of claim 17, wherein the time when the portion of biometric sensor information was captured equates to the time when the portion of biometric sensor information was received.

19. The system of claim 11, wherein the program instructions further direct the processing system to:
present a graphical representation of portions of the biometric sensor information with contemporaneous portions of the media.

20. A method of selecting cloud computing providers from a plurality of cloud computing providers, the method comprising:
obtaining one or more parameters for a computing job;
obtaining a cost bid based on the one or more parameters from each of the plurality of cloud computing providers;
selecting one or more cloud computing providers of the plurality of cloud computing providers based on the cost bids.
